(19) United States
(12) Patent Application Publication
(10) Pub. No.: US 2021/0361552 A1
(43) Pub. Date: Nov. 25, 2021

(54) 24-karat Edible Gold Glitter Lipscrub

(71) Applicant: Catrina Medina, Garnerville, NY (US)

(72) Inventor: Catrina Medina, Garnerville, NY (US)

(21) Appl. No.: 16/878,816

(22) Filed: May 20, 2020

(51) Int. Cl. A61K 8/60; A61K 8/02; A61K 8/19; A61Q 19/00; A61K 8/92

(52) U.S. Cl. CPC A61K 8/60; A61K 8/0225; A61K 8/922; A61Q 19/001; A61K 8/19

(57) ABSTRACT

The present invention is the combined fusion of the sugar and oils based lip scrub with the 24-karat edible Gold dust addictive to enhance the visual purpose of the scrub and provide a hint of sparkle to the lips. The focus of the invention is on the joining of the lip scrub with the 24-karat edible Gold dust addictive.

### CROSS REFERENCES TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

### STATEMENT REGARDING PRIOR DISCLOSURES BY THE INVENTOR OR A JOINT INVENTOR

Not Applicable

### BACKGROUND OF THE INVENTION

### (1) Technical Field

The present invention relates to the fusion between a cosmetic invention and an addictive.

### (2) Background Technique

Purpose of a lip scrub is to remove dead skin cells that tend to dull the skin. The dead skin cells on excessively dry lips can begin to flake off which results in peeling or chapped lips. In order to achieve supple moisturized lips a scrub can be added to a daily beauty routine.

The addictive that will be mentioned in the summary of the invention is 24-karat edible gold dust. The dust is of a light-reflective gold that contains non-toxic ingredients. Edible gold is chemically inert and will not react inside the body. Edible gold has an E Number which is E175 and is widely recognized as a food additive. 24-karat edible gold dust is the purest, softest form to digest since it has very few impurities.

### SUMMARY OF THE INVENTION

The present invention fuses the two above mentioned techniques using a lip scrub to achieve moisturized soft lips with an added sparkle to the lips from the 24-karat edible gold dust addictive.

### BRIEF DESCRIPTION OF THE SEVERAL VIEWS OF THE DRAWINGS

Not Applicable

### DETAILED DESCRIPTION OF THE INVENTION

The invention contains: (1) granulated sugar which is a pure sugar that has been crystallized and centrifuged and then sent through a granulator where the crystals are dried, separated and screened. (2) a moisturizing agent of coconut oil which is a fatty oil or semisolid fat extracted from fresh coconuts. (3) vitamin e oil which is both a nutrient and antioxidant. Its a prevalent ingredient in both natural and store-bought cosmetics since it acts as a natural stabilizer and can prolong the shelf life of a cosmetic products. (4) essential oils in lip scrub is used for aromatherapy purposes from relieving stress to uplifting and rejuvenating your body. (5) the 24-karat edible gold dust addictive is strictly for appearances in the scrub itself and on the lips since it is flavorless, odorless and nontoxic. The sparkle and light-reflective gold dust provides a visual satisfaction use.

1. The combined fusion of the sugar and oils based lip scrub with the 24-karat edible Gold dust addictive to enhance the visual purpose of the scrub and provide a hint of sparkle to the lips.
